(19) United States
(12) Patent Application Publication
(10) Pub. No.: US 2022/0411739 A1
(43) Pub. Date: Dec. 29, 2022

(54) GENETICALLY MODIFIED CELL PRODUCTION SYSTEM

(71) Applicants: Noile-Immune Biotech Inc., Minato-ku, Tokyo (JP); SHIBUYA CORPORATION, Kanazawa-shi, Ishikawa (JP)

(72) Inventors: Kenji YONEDA, Kanazawa-shi, Ishikawa (JP); Naoya DEGUCHI, Nonoichi-shi, Ishikawa (JP); Noriaki NISHIMURA, Kanazawa-shi, Ishikawa (JP)

(21) Appl. No.: 17/771,255

(22) PCT Filed: Sep. 30, 2020
(86) PCT No.: PCT/JP2020/037123

§ 371 Date: Apr. 22, 2022

(30) Foreign Application Priority Data
Oct. 25, 2019 (JP) 2019-194061

(51) Int. Cl. C12M 1/42; C12M 1/00; C12M 1/32

(52) U.S. Cl. CPC C12M 35/08; C12M 23/58; C12M 33/06; C12M 23/40; C12M 39/00

(57) ABSTRACT

A genetically modified cell that can be mass-produced efficiently. The genetically modified cell production system includes a cell-processing isolator for seeding a T cell in a well plate (culture vessel) a cell culture incubator (cell storage) for holding the culture vessel in which the T cell has been seeded and culturing the T cell a virus-processing isolator (nucleic acid preparation isolator) for providing, to the well plate, a virus having a nucleic acid containing a gene to be introduced into a cell a virus storage (nucleic acid storage) for holding the well plate from the virus-processing isolator and a virus infection isolator (nucleic acid introduction isolator) for introducing the nucleic acid into the T cell by infecting the T cell with the virus.

### TECHNICAL FIELD

The present invention relates to a genetically modified cell production system, and more particularly, it relates to a genetically modified cell production system for producing a genetically modified cell having a gene modified by introducing a nucleic acid into a cell.

### BACKGROUND ART

Today, for the purpose of treating cancer or the like, a CAR-T cell containing a T cell having a chimeric antigen receptor (CAR) expressed therein is administered to a patient.

For producing such a CAR-T cell, a method for producing a genetically modified cell having a gene modified by introducing a nucleic acid into a cell is utilized, and specifically, an operation for infecting a T cell collected from a patient with a virus having a nucleic acid containing a gene encoding a CAR to introduce the nucleic acid into the T cell and express the CAR therein is necessary (Patent Literature 1).

Such a method for producing a genetically modified cell by introducing a nucleic acid into a cell is applied to production of various cells, and in addition, a nucleic acid is introduced into a stem cell or a progenitor cell.

### PRIOR ART DOCUMENTS

### Patent Literature

Patent Literature 1: Japanese Patent No. 6161098

### SUMMARY OF INVENTION

### Problems to be Solved by the Invention

A treatment with a genetically modified cell produced by introducing a nucleic acid into a cell is predicted to increase in demand in the future, but such an operation for producing a genetically modified cell is currently manually performed in one isolator, and hence takes time, and is not suitable for mass culture.

In consideration of these problems, the present invention provides a genetically modified cell production system capable of producing a genetically modified cell having a nucleic acid introduced therein efficiently and in a large amount.

### Means for Solving the Problems

Specifically, a genetically modified cell production system according to an invention of claim 1 is a genetically modified cell production system for producing a genetically modified cell having a gene modified by introducing a nucleic acid into a cell, characterized by including: a cell-processing isolator for seeding a cell in a culture vessel with sterile conditions kept inside, and a cell storage, configured to be connectable/disconnectable to/from the cell-processing isolator with the sterile conditions kept, for holding the culture vessel in which the cell has been seeded to transfer the cell; a nucleic acid preparation isolator for preparing a nucleic acid containing a gene to be introduced into the cell with sterile conditions kept inside, and a nucleic acid storage, configured to be connectable/disconnectable to/from the nucleic acid preparation isolator with the sterile conditions kept, for holding the prepared nucleic acid for transfer; and a nucleic acid introduction isolator, configured to have sterile conditions kept inside, and be connectable/disconnectable simultaneously to/from the cell storage and the nucleic acid storage with the sterile conditions kept, for introducing the nucleic acid having been transported from the nucleic acid storage into the cell having been transported from the cell storage.

### Advantageous Effects of Invention

According to the invention, an operation for seeding and culturing a cell in a culture vessel, an operation for preparing a nucleic acid containing a gene, and an operation for introducing the nucleic acid into the cultured cell can be performed in parallel with isolators kept under sterile conditions inside, and hence a genetically modified cell can be produced efficiently and in a large amount.

Besides, since the cell storage and the nucleic acid storage are included, a cell and a nucleic acid can be transferred among isolators with the sterile conditions kept.

Furthermore, since the cell storage and the nucleic acid storage can be simultaneously connected to the nucleic acid introduction isolator, a cell can be taken out of the cell storage and a nucleic acid can be taken out of the nucleic acid storage simultaneously in the nucleic acid introduction isolator, and thus, an efficient operation can be performed.

### BRIEF DESCRIPTION OF DRAWING

FIG. 1 is a plan view of a cell processing facility in which a genetically modified cell production system is installed.

### MODE FOR CARRYING OUT THE INVENTION

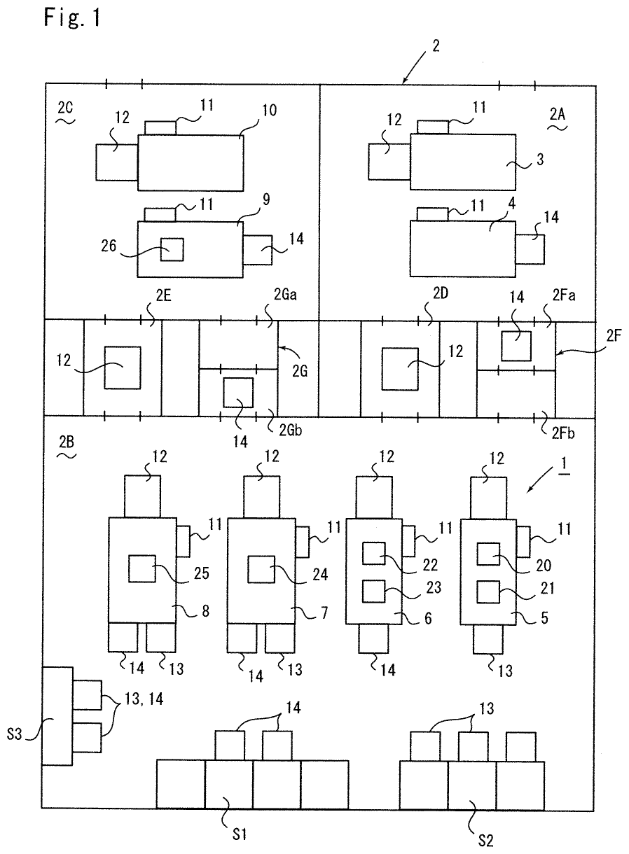

An example illustrated in the accompanying drawing will now be described, and FIG. 1 is a plan view of a cell processing facility 2 in which a genetically modified cell production system 1 of the present invention is installed.

In the genetically modified cell production system 1 of the present example, a CAR-T cell is produced as a genetically modified cell, and the CAR-T cell is a T cell in which a chimeric antigen receptor (CAR) is expressed, and is used in a treatment of cancer and the like in recent years.

As a method for producing the CAR-T cell, a method for producing a genetically modified cell having a gene modified by introducing a nucleic acid into a cell is employed, and although the details are not described because the method is specifically described in Patent Literature 1 mentioned above, a T cell is cultured as a cell collected from a patient, a virus having a nucleic acid containing a gene encoding (expressing) a CAR is supplied to a well plate corresponding to a culture vessel, and then, the cultured T cell is seeded in a well plate corresponding to a culture vessel to which the virus has been supplied.

In this manner, the T cell is infected with the virus, and the nucleic acid (gene) is introduced from the virus into the T cell so that the chimeric antigen receptor can be expressed. As such, a method known as what is called a gene transfer method with a virus vector is employed.

Besides, in the present example, in seeding the T cell collected from the patient in the well plate, an activator is precedently supplied to the well plate to promote activation of the T cell.

Here, the well plate used in the present example is a resin vessel in which a plurality of concave wells is formed, and cells are seeded in each well to be cultured. Besides, in the present genetically modified cell production system, a culture vessel different from the well plate, such as a petri dish or a flask, can be applied.

Furthermore, by using the genetically modified cell production system 1 of the present example, other types of genetically modified cell can be produced by performing operations similar to production procedures for the CAR-T cell described below.

Here, a genetically modified cell refers to a cell in which a foreign nucleic acid has been introduced, is not limited to one obtained by editing an intrinsic gene sequence, but encompasses a cell in which a nucleic acid temporarily expressing a protein or a peptide without changing an intrinsic gene sequence, or a functional nucleic acid regulating transcription or expression of a protein or the like has been introduced. Besides, a receptor to be expressed is not limited to the chimeric antigen receptor but can be a hormone receptor, a photoreceptor or the like.

Examples of a cell that can be dealt with in the genetically modified cell production system 1 include, in addition to the T cell, immune cells such as other lymphoid cells (such as a natural killer cell (NK cell), or a B cell), an antigen presenting cell (such as a monocyte, a macrophage, or a dendritic cell), and a granulocyte (such as a neutrophil, an eosinophil, a basophil, or a mast cell).

Other examples of the cell include, in addition to the above-described immune cells, a stem cell (a hematopoietic stem cell, a somatic stem cell, a totipotent stem cell, a pluripotent stem cell, a fetal stem cell, an embryonic stem cell, a mesenchymal stem cell, or an induced pluripotent stem cell), a progenitor cell (such as a satellite cell, a neural progenitor cell, a bone marrow stromal cell, a pancreatic progenitor cell, an angioblast, or an endothelial progenitor cell), and a differentiated cell (such as an epithelial cell, a cardiac myocyte, a fibroblast, or a chondrocyte).

On the other hand, the nucleic acid may be naturally derived or artificially synthesized, and specifically, can be DNA, RNA, siRNA, miRNA, ami-RNA, shRNA, dsRNA or the like. Alternatively, the nucleic acid can be a DNA encoding, for example, a protein or a polypeptide. Further alternatively, the nucleic acid may be a DNA encoding siRNA, miRNA, ami-RNA, shRNA, dsRNA or the like.

In the present example, the gene transfer method with a virus vector for producing the CAR-T cell by infecting the T cell with a virus is employed, but as another method for introducing a nucleic acid (gene) into a cell, a transfection method (such as a calcium phosphate method, a lipofection method, or an electroporation method), or a microinjection method can be employed.

The cell processing facility 2 of the present example includes a material supply chamber 2A for preparing a material to be used in the production of the CAR-T cell, a cell manipulation chamber 2B for producing the CAR-T cell, and a cell collection chamber 2C for collecting the cultured CAR-T cell.

Interior spaces of the material supply chamber 2A, the cell manipulation chamber 2B, and the cell collection chamber 2C are partitioned in an airtight manner from an exterior space of the cell processing facility 2, and are kept at a higher cleanliness than in the exterior space, and among these chambers, the cell manipulation chamber 2B is kept at a higher cleanliness than in, and at a positive pressure with respect to the material supply chamber 2A and the cell collection chamber 2C.

In the cell manipulation chamber 2B, it is necessary to inhibit inflow of an external atmosphere, and to prevent outflow of the virus to the outside, and between the cell manipulation chamber 2B and the material supply chamber 2A, and between the cell manipulation chamber 2B and the cell collection chamber 2C, material passing rooms 2D and 2E through which a transferring vessel 12 holding a material described below is transferred, and cell passing rooms 2F and 2G through which a cell culture incubator 14 holding a cell is transferred are provided.

The material passing room 2D is provided with openable opening/closing doors respectively communicating with the material supply chamber 2A and the cell manipulation chamber 2B, and the inside of the material passing room 2D is set at a higher air pressure than in the material supply chamber 2A, and at a lower air pressure than in the cell manipulation chamber 2B.

Similarly, the material passing room 2E is provided with openable opening/closing doors respectively communicating with the cell manipulation chamber 2B and the cell collection chamber 2C, and the inside of the material passing room 2E is set at a lower air pressure than in the cell manipulation chamber 2B, and at a higher air pressure than in the cell collection chamber 2C.

Furthermore, the material passing rooms 2D and 2E are provided with decontamination device for decontaminating the interior spaces of these rooms with a decontamination medium such as hydrogen peroxide vapor, and thus, the outer surface of the transferring vessel 12 can be decontaminated.

The cell passing room 2F is provided with openable opening/closing doors respectively communicating with the material supply chamber 2A and the cell manipulation chamber 2B, and a partition having an opening/closing door is provided therein for partitioning into a space 2Fa on a side of the material supply chamber 2A, and a space 2Fb on a side of the cell manipulation chamber 2B.

The space 2Fa on the side of the material supply chamber 2A is set at a higher air pressure than in the material supply chamber 2A, and on the contrary, the space 2Fb on the side of the cell manipulation chamber 2B is set at a higher air pressure than in the space 2Fa on the side of the material supply chamber 2A, and at a lower air pressure than in the cell manipulation chamber 2B.

Similarly, the cell passing room 2G is also partitioned with a partition, a space 2Ga on a side of the cell collection chamber 2C is set at a higher air pressure than in the cell collection chamber 2C, and on the contrary, a space 2Gb on a side of the cell manipulation chamber 2B is set at a higher air pressure than in the space 2Ga on the side of the cell collection chamber 2C, and at a lower air pressure than in the cell manipulation chamber 2B.

In the material supply chamber 2A, a material supply isolator 3 for preparing a material to be used in the production of the CAR-T cell, and a cell preparation isolator 4 for preparing the T cell to be cultured are provided.

In the cell manipulation chamber 2B, a virus-processing isolator 5 corresponding to a nucleic acid preparation isolator for supplying an adhesion factor or a virus to a well plate, a cell-processing isolator 6 for supplying an activator to the well plate and seeding the T cell, and a first virus infection isolator 7 corresponding to a nucleic acid introduction isolator for seeding the T cell in the well plate where the virus has been supplied are provided.

In the present example, in case that virus infection in the first virus infection isolator 7 is insufficient, a second virus infection isolator 8 corresponding to a nucleic acid introduction isolator for seeding the T cell having been processed in the first virus infection isolator again in the well plate in which the virus has been supplied is also provided, but only one virus infection isolator may be provided.

Besides, in the cell manipulation chamber 2B, a culture station S1 for culturing the T cell, a storage station S2 for storing the well plate in which the virus has been supplied, and a decontamination station S3 corresponding to a decontamination device for decontaminating a virus storage 13 corresponding to a nucleic acid storage and a cell culture incubator 14 already used are provided.

In the cell collection chamber 2C, a cell collection isolator 9 for collecting the produced CAR-T cell, and a material collection isolator 10 for collecting and discarding used materials are provided.

In the cell manipulation chamber 2 of the genetically modified cell production system 1 having the above-described structure, various processing devices provided in the respective isolators are controlled by control device not shown, and thus, operations in the respective isolators can be automatically performed.

In the present example, the respective isolators are configured to be kept in sterile conditions inside, and hence the operations for producing the CAR-T cell can be performed under a sterile environment.

The isolators themselves are conventionally known, and hence are not described in detail, and are configured to keep sterile conditions of the interior spaces, and are provided, on side surfaces, with gloves to be worn by an operator to perform an operation in the interior space.

Furthermore, each isolator is provided with a decontamination device 11 for decontaminating the interior space with a decontamination medium such as hydrogen peroxide vapor, so that the interior space can be decontaminated with the decontamination device 11 when the production of the CAR-T cell for a given patient is completed.

Besides, in the genetically modified cell production system 1 of the present example, a plurality of transferring vessels 12, virus storages 13 and cell culture incubators 14 connectable/disconnectable to/from each of the isolators with the sterile conditions kept are provided for transferring the T cell and the virus between the isolators.

The transferring vessel 12 is used for transferring the material or the virus prepared in the material supply isolator 3 to each of the other isolators, and the transferring vessel 12 is provided to be connected to the material supply isolator 3, the virus-processing isolator 5, the cell-processing isolator 6, and the first and second virus infection isolators 7 and 8.

The virus storage 13 is used as the nucleic acid storage according to the present invention, is used to store the well plate to which the virus has been supplied in the virus-processing isolator 5 and to transfer it to each of the other isolators, and has a temperature control function for storing the well plate held therein at a desired low temperature.

The virus storage 13 is provided to be connected to the virus-processing isolator 5 and the first and second virus infection isolators 7 and 8, and to be connected to the storage station S2 and the decontamination station S3.

The cell culture incubator 14 is used as the cell storage member according to the present invention, and is used to store the well plate in which the cell has been seeded in the cell-processing isolator 6, and to transfer it to another isolator. As described below, the cell culture incubator 14 is configured as an incubator for culturing the T cell, and has functions to control a temperature, a humidity, a carbon dioxide gas concentration, and the like.

The cell culture incubator 14 is provided to be connected to the cell preparation isolator 4, the cell-processing isolator 6, the first and second virus infection isolators 7 and 8, and the cell collection isolator 9, and to be connected to the culture station S1 and the decontamination station S3.

The virus storage 13 and the cell culture incubator 14 used in the cell manipulation chamber 2B are configured to be capable of self-propelling within the cell manipulation chamber 2B, and materials can be automatically transferred by controlling these by the control device.

Connecting means for connecting the transferring vessel 12, the virus storage 13 and the cell culture incubator 14 to each of the isolators is conventionally known, and hence is not described in detail, and description will be given here by exemplifying connecting means for connecting the material supply isolator 3 and the transferring vessel 12 to each other.

In each of the material supply isolator 3 and the transferring vessel 12, an opening that can be opened/closed by an opening/closing door is formed, and a connecting member is provided to surround the opening.

When the connecting members are connected to each other, a space partitioned from the outside is formed between the openings, and the space can be decontaminated by supplying the decontamination medium to the space from the decontamination device 11.

When the decontamination is completed, the opening/closing doors of the material supply isolator 3 and the transferring vessels 12 are opened, and as a result, the interior space of the material supply isolator 3 and the interior space of the transferring vessel 12 are communicated with each other with the sterile conditions kept, and then, a material or the like can be moved.

Now, the respective isolators and the operations performed in the respective isolators will be described to describe production procedures for the CAR-T cell.

First, the material supply isolator 3 provided in the material supply chamber 2A is an isolator for performing a preparation operation for distributing materials necessary for the production of the CAR-T cell, and a virus having a nucleic acid to be introduced into a cell to the virus-processing isolator 5, the cell-processing isolator 6, and the first and second virus infection isolators 7 and 8 of the cell manipulation chamber 2B. The transferring vessel 12 is connectable/disconnectable to/from the material supply isolator 3 with the sterile conditions kept.

The materials include, in addition to a well plate, a vessel for holding a liquid, and a pipette used in a dispensing device, an aspirator or the like.

Regarding an operation for transferring a material from the material supply isolator 3 to another isolator, an operator first transports the material from the outside of the cell processing facility 2 into the material supply chamber 2A, closes the door of the material supply chamber 2A, and then holds the material in the material supply isolator 3.

Subsequently, if the material is packaged, the package is removed, the isolator is filled with a decontamination medium with the decontamination device 11 provided in the material supply isolator 3, and thus, the outer surface of the material is decontaminated.

When the decontamination of the material is thus completed, the material is held in the transferring vessel 12 precedently connected, and then, the transferring vessel 12 is detached from the material supply isolator 3 to be placed in the material passing room 2D provided between the material supply chamber 2A and the cell manipulation chamber 2B. The door of the material passing room 2D to the cell manipulation chamber 2B is closed from the beginning, and after placing the transferring vessel 12 in the material passing room 2D, the door to the material supply chamber 2A is also closed.

Then, the interior space of the material passing room 2D is filled with the decontamination medium, so as to decontaminate the outer surface of the transferring vessel 12. Thereafter, the door of the material passing room 2D to the cell manipulation chamber 2B is opened, and thus, the transferring vessel 12 can be moved into the cell manipulation chamber 2B to be connected to a desired isolator.

Next, the cell preparation isolator 4 provided in the material supply chamber 2A is an isolator for performing an operation for transporting a cell to be used in the production of the CAR-T cell to the cell-processing isolator 6 of the cell manipulation chamber 2B. The cell culture incubator 14 is connectable/disconnectable to/from the cell preparation isolator 4 with the sterile conditions kept.

Regarding an operation for transferring the T cell from the cell preparation isolator 4 to the cell-processing isolator 6, a vessel or the like holding the T cell is first transported from the outside of the cell processing facility 2, the vessel or the like is held in the cell preparation isolator 4, and in the cell preparation isolator 4, the T cell is prepared into a suspension and held in a centrifuge tube.

The centrifuge tube is held in the cell culture incubator 14, and then the cell culture incubator 14 is detached from the cell preparation isolator 4 to be placed in the cell passing room 2F provided between the material supply chamber 2A and the cell manipulation chamber 2B.

The cell culture incubator 14 is first held in the space 2Fa on the side of the material supply chamber 2A, and with the opening/closing door of the space 2Fa to the material supply chamber 2A and the opening/closing door to the space 2Fb closed, the outer surface of the cell culture incubator 14 is cleaned with an alcohol or the like.

Specifically, in consideration of influence on the cell of time necessary for the decontamination, the cell culture incubator 4 is not decontaminated with the decontamination medium such as hydrogen peroxide vapor but is sterilized on the outer surface with a bactericide such as an alcohol.

Thereafter, the opening/closing door of the space 2Fa to the space 2Fb is opened, the cell culture incubator 14 is moved to the space 2Fb on the side of the cell manipulation chamber 2B, and after closing the opening/closing door, the opening/closing door of the space 2Fb to the cell manipulation chamber 2B is opened to move the cell culture incubator 14 from the space 2Fb to the cell manipulation chamber 2B.

Here, although the opening/closing doors are opened/closed in these operations, the entry of the external atmosphere to the cell manipulation chamber 2B can be prevented as much as possible because a pressure difference is provided between the rooms.

The virus-processing isolator 5 provided in the cell manipulation chamber 2B is used as a nucleic acid preparation isolator for preparing a nucleic acid containing a gene to be introduced into a cell, and in the present example, is an isolator for supplying an adhesion factor and the virus containing the nucleic acid to each well of the well plate.

The transferring vessel 12 and the virus storage 13 are connectable/disconnectable to/from the virus-processing isolator 5 with the sterile conditions kept. Furthermore, a preparation operation performed in the nucleic acid preparation isolator can be an operation for preparing a nucleic acid, and a preparation operation for introducing, into a virus, the nucleic acid to be introduced into the cell.

In the virus-processing isolator 5, adhesion factor supply device 20 for supplying the adhesion factor to each well of the well plate, and virus supply device 21 for supplying the virus to each well of the well plate are provided.

Although the structure of the adhesion factor supply device 20 is not described in detail, for automatically performing operations described below, dispensing device for dispensing a prescribed amount of the adhesion factor to each well of the well plate, aspirator device for removing a liquid from each well, cleaning solution supply device for supplying a cleaning solution to each well, moving device for moving these means relatively to the well plate, and the like are provided, and these means are controlled by the above-described control device.

Besides, the virus supply device 21 similarly includes, for automatically performing operations described below, dispensing device for dispensing a prescribed amount of the virus to each well of the well plate, a centrifuge for centrifuging the whole well plate with the virus dispensed thereinto, aspirator device for removing a liquid from each well, cleaning solution supply device for supplying a cleaning solution to each well, moving device for moving these devices relatively to the well plate, and the like, and these devices are also controlled by the control device.

Now, an operation for supplying a virus to a well plate with the virus-processing isolator 5 will be described.

First, an operation for supplying an adhesion factor to the well plate is precedently performed with the adhesion factor supply device 20 of the virus-processing isolator 5.

The transferring vessel 12 is first connected to the virus-processing isolator 5 to transport the well plate and other materials thereto, and a vessel holding the adhesion factor having adhesive action is supplied.

Subsequently, the adhesion factor supply device 20 is activated to aspirate the adhesion factor from the vessel holding the adhesion factor, dispenses the adhesion factor to each well of the well plate, and then, holds the resultant for a prescribed period of time. Thus, the adhesion factor is supplied to each well.

Next, the virus supply device 21 of the virus-processing isolator 5 supplies the virus to the well plate in which the adhesion factor has been supplied.

After the adhesion factor supply device 20 supplies the adhesion factor, the well plate is moved alternately between the aspirator device and the cleaning solution supply device of the adhesion factor supply device 20 to perform removing an unnecessary portion of the adhesion factor from each well and cleaning.

The thus obtained well plate in which the adhesion factor has been supplied is moved to the virus supply device 21, and the virus supply device 21 aspirates the virus from the vessel holding the virus to dispense a prescribed amount of the virus into each well of the well plate.

Thereafter, the well plate is charged in the centrifuge in a state where the virus has been dispensed into each well, and the well plate is centrifuged for about 2 hours to fix the virus in each well.

Subsequently, the well plate is moved alternately between the aspirator device and the cleaning solution supply device to clean the wells by removing the adhesion factor from each well, and further, by repeatedly supplying and removing the cleaning solution to and from the wells.

In this manner, a well plate in which the virus having the nucleic acid is adsorbed in each well is obtained, in other words, the nucleic acid is prepared in the nucleic acid preparation isolator.

Thereafter, the cleaning solution is dispensed into each well again to prevent the virus from drying, and the well plate into which the cleaning solution has been dispensed is held in the virus storage 13 connected to the virus-processing isolator 5.

Thereafter, the virus storage 13 is detached from the virus-processing isolator 5, and is moved and connected to the storage station S2 adjacently provided. As described above, the virus storage 13 has the temperature control function, and in the storage station S2, necessary electric power or the like is supplied to the virus storage 13.

Then, the inside of the virus storage 13 is kept at a prescribed low temperature, and the virus supplied to the well plate is stored so as not to be activated more than necessary.

The cell-processing isolator 6 provided in the cell manipulation chamber 2B is an isolator for performing an operation for seeding a T cell in a well plate, and the transferring vessel 12 and the cell culture incubator 14 are connectable/disconnectable thereto/therefrom.

In the cell-processing isolator 6, activator supply device 22 for supplying an activator to a well plate, and first seeding device 23 for seeding the T cell in the well plate are provided.

The activator supply device 22 has a structure similar to that of the adhesion factor supply device 20 provided in the virus-processing isolator 5, and hence is not described in detail.

The first seeding device 23 includes counting device for counting the number of T cells in a T cell suspension, culture fluid supply device for diluting the T cell suspension with a culture fluid, dispensing device for dispensing the diluted T cell suspension into the well plate, aspirator device for removing a liquid from each well, cleaning solution supply device for dispensing a cleaning solution into each well, moving device for moving these devices relatively to the well plate, and the like, and these devices are controlled by control device.

An operation for seeding the T cell in the well plate with the cell-processing isolator 6 will now be described.

First, the transferring vessel 12 is connected to the cell-processing isolator 6, the well plate and other materials are transported thereto, the cell culture incubator 14 is connected thereto, and a centrifuge tube holding the T cell suspension is transported thereto.

Then, the activator supply device 22 is first activated to supply the activator to the well plate.

When the activator is thus supplied to the well plate, the aspirator device and the cleaning solution supply device of the first seeding device 23 cleans the well plate by supplying and removing a cleaning solution to and from each well of the well plate.

Next, the dispensing device aspirates a small amount of the T cell suspension from the centrifuge tube holding the T cell suspension to count the number of cells, and calculates, in accordance with the number of cells, an amount of a culture fluid necessary for attaining a prescribed concentration of the T cell when the T cell suspension is diluted with the culture fluid.

Then, after the culture fluid supply device dispenses the calculated amount of the culture fluid into a desired centrifuge tube, the dispensing device aspirates the T cell suspension from the centrifuge tube holding the T cell suspension to supply it to the centrifuge tube holding the culture fluid to prepare a T cell suspension having a prescribed concentration.

Thereafter, the dispensing device dispenses the thus prepared T cell suspension into the wells of the well plate in which the activator has been supplied, and thus, a T cell well plate in which the T cell has been seeded is obtained.

The well plate in which the T cell has been thus seeded is held in the cell culture incubator 14 connected to the cell-processing isolator 6, and then is moved to the culture station S1 provided in the cell manipulation chamber 2B and the cell culture incubator 14 is connected thereto.

Here, the cell culture incubator 14 is configured as an incubator, and when the cell culture incubator 14 is connected to the culture station S1, electric power and a carbon dioxide gas are supplied to the cell culture incubator 14.

In the present example, the T cell seeded in the well plate is cultured in the cell culture incubator 14 for about 72 hours, during which the seeded T cell is activated by the activator supplied to the well while adhering to the well.

The first virus infection isolator 7 provided in the cell manipulation chamber 2B is used for first introducing a nucleic acid to the cell cultured in the cell culture incubator 14, and specifically is an isolator for performing operations for collecting the T cell cultured in the well plate and seeding the resultant in a well plate where the virus has been supplied, and the transferring vessel 12, the virus storage 13, and the cell culture incubator 14 are connectable thereto.

In the first virus infection isolator 7, second seeding device 24 that takes out the T cell from the well plate in which the T cell is cultured, and seeds the T cell in the well plate in which the virus has been supplied to infect the T cell with the virus is provided.

Although the structure of the second seeding device 24 is not described in detail, it basically has a structure similar to that of the first seeding device 23 of the cell-processing isolator 6, and is controlled by the control device.

An operation for infecting the T cell with the virus with the first virus infection isolator 7 will now be described.

First, the cell culture incubator 14 is connected to the first virus infection isolator 7 to transport the well plate in which the T cell is cultured thereto, and the virus storage 13 is connected thereto to transport the well plate in which the virus has been supplied thereto.

In particular, the first virus infection isolator 7 is provided with a connecting portion to be connected to the cell culture incubator 14, and a connecting portion to be connected to the virus storage 13, and the cell culture incubator 14 and the virus storage 13 can be connected simultaneously or at different timing.

At this point, when the cell culture incubator 14 and the virus storage 13 are connected to the first virus infection isolator 7 simultaneously, decontamination performed in connecting these, and taking out of the well plates of the cell and the virus stored therein can be performed simultaneously, and hence an efficient operation can be performed.

Then, the second seeding device 24 collects the T cell from each well of the well plate in which the T cell is cultured to prepare a T cell suspension in a centrifuge tube, and samples a small amount from the centrifuge tube for counting the number of cells.

Next, based on the result of counting the number of cells, an amount of a culture fluid necessary for attaining a prescribed concentration of the T cell in the T cell suspension is calculated, the calculated amount of the culture fluid is held in another centrifuge tube, and then the T cell suspension of the well plate is added to the centrifuge tube.

Subsequently, after removing the cleaning solution from each well of the well plate in which the virus transported from the virus storage 13 has been supplied, the T cell suspension is dispensed into each well of the well plate.

When the T cell is thus seeded in the well plate in which the virus has been supplied, the cell culture incubator 14 is connected to the first virus infection isolator 7 to hold the well plate therein, and then the cell culture incubator 14 is detached from the first virus infection isolator 7 to be moved and connected to the culture station S1.

Then, in the culture station S1, electric power and a carbon dioxide gas for operating the cell culture incubator 14 configured as the incubator are supplied, and the T cell is infected with the virus by holding the well plate in the cell culture incubator 14 for a prescribed period of time.

The second virus infection isolator 8 provided in the cell manipulation chamber 2B is used for introducing again the nucleic acid to the cell having been processed in the first virus infection isolator 7, and specifically, is an isolator for seeding again the T cell having been processed in the first virus infection isolator 7, in the well plate in which the virus has been supplied, and the transferring vessel 12, the virus storage 13, and the cell culture incubator 14 are connectable thereto.

Also, the second virus infection isolator 8 has a connecting portion of the cell culture incubator 14 and a connecting portion of the virus storage 13, and hence the cell culture incubator 14 and the virus storage 13 can be connected thereto simultaneously, and thus an efficient operation can be performed.

In the second virus infection isolator 8, third seeding device 25 that takes out the T cell from the well plate in which the T cell infected with the virus, and seeds the T cell in the well plate in which the virus has been supplied to infect again the T cell with the virus is provided.

Although the structure of the third seeding device 25 is not described in detail, it basically has a mechanism similar to the first seeding device 23 of the cell-processing isolator 6 and the second seeding device 24 of the first virus infection isolator 7, and is provided to be controlled by the control device.

An operation for seeding again the T cell having been infected with the virus by the second virus infection isolator 8 in the well plate in which the virus has been prepared will now be described.

First, the cell culture incubator 14 is connected to the second virus infection isolator 8 to transport the well plate in which the T cell has been infected with the virus thereto, and the virus storage 13 is connected thereto to transport the well plate in which the virus has been supplied thereto.

Then, the third seeding device 25 collects the T cell from the well plate in which the T cell has been infected with the virus, and dispenses the T cell into each well of the well plate transported from the virus storage 13 in which the virus has been supplied, so as to infect the T cell with the virus again.

After the T cell is thus seeded again in the well plate in which the virus has been supplied, the well plate is held in the cell culture incubator 14 connected to the second virus infection isolator 8, and the resultant is allowed to stand still for about 2 hours to further infect the T cell with the virus.

Next, the cell culture incubator 14 is detached from the second virus infection isolator 8 to be moved to the cell collection isolator 9 of the cell collection chamber 2C.

The cell collection isolator 9 provided in the cell collection chamber 2C is an isolator for collecting the T cell in which a chimeric antigen receptor (CAR) has been expressed by introducing the gene through virus infection performed in the second virus infection isolator 8, namely, the CAR-T cell, and the cell culture incubator 14 can be connected thereto.

In the cell collection isolator 9, cell collecting device 26 including a dispensing device, an aspirator and the like for collecting the CAR-T cell from the well plate having been transported from the cell culture incubator 14 is provided to automatically perform an operation described below.

An operation for collecting the CAR-T cell from the well plate with the cell collection isolator 9 will now be described.

First, the cell culture incubator 14 holding the well plate is detached from the culture station S1 of the cell manipulation chamber 2B, and the cell culture incubator 14 is held in the cell passing room 2G provided between the cell manipulation chamber 2B and the cell collection chamber 2C.

First, the opening/closing door of the space 2Gb to the space 2Ga is closed, and after holding the cell culture incubator 14 in the space 2Gb on the side of the cell manipulation chamber 2B, the opening/closing door of the space 2Gb to the cell manipulation chamber 2B is also closed, and the outer surface of the cell culture incubator 14 is cleaned with an alcohol or the like in the same manner as in the cell passing room 2F.

Thereafter, the opening/closing door of the space 2Gb to the space 2Ga is opened, the cell culture incubator 14 is moved to the space 2Ga on the side of the cell collection chamber 2C, and is moved further to the cell collection chamber 2C to be connected to the cell collection isolator 9.

When the well plate is transported to the cell collection isolator 9, the cell collecting device 26 performs an operation for collecting the CAR-T cells from the respective wells to gather these in one collection vessel.

Then, after the CAR-T cells are gathered in one vessel, the vessel is sealed, and then, an operator takes the vessel out of the cell collection isolator 9, and carries it out from the cell collection chamber 2C to the outside of the cell processing facility 2. The CAR-T cell thus produced can be further subjected to expanding culture in an external facility.

The material collection isolator 10 provided in the cell collection chamber 2C is an isolator for collecting, for discarding, the materials such as the well plate and other vessels used in the respective isolators in a waste bag or the like, and the transferring vessel 12 can be connected thereto.

Here, the transferring vessel 12 is configured to transfer, when connected to each isolator in the cell manipulation chamber 2B, the materials held therein in the material supply isolator 3 to the isolator, and to receive used materials.

When the processing in each isolator is completed, the transferring vessel 12 is detached from each isolator, and then is held in the material passing room 2E provided between the cell manipulation chamber 2B and the cell collection chamber 2C, and the outer surface thereof is decontaminated with the decontamination medium.

Thereafter, the transferring vessel 12 is moved to the cell collection chamber 2C, and when it is connected to the material collection isolator 10, an operator collects the used material held in the transferring vessel 12 in a waste bag or the like, and decontaminates the outer surface of the bag again after completing the collection.

Then, when a prescribed amount of the materials is collected in the bag, the bag is sealed, and then, an operator takes the bag out of the material collection isolator 10 and carries it out from the cell collection chamber 2C to the outside of the cell processing facility 2.

In addition, the transferring vessel 12, the virus storage 13, and the cell culture incubator 14 used in the production of the CAR-T cell are configured to be connected, after the use, to the decontamination station S3 provided in the cell manipulation chamber 2B.

When the transferring vessel 12, the virus storage 13, and the cell culture incubator 14 are connected, the decontamination station S3 performs decontamination by supplying the decontamination medium such as hydrogen peroxide vapor to the interior spaces thereof.

Besides, also the inside of each of the isolators is decontaminated with the decontamination device 11.

According to the present example, the operation for producing a CAR-T cell including a plurality of procedures can be performed in parallel with a plurality of isolators, and hence the CAR-T cell can be produced efficiently and in a large amount.

In particular, the first and second virus infection isolators 7 and 8 have the structure in which the virus storage 13 and the cell culture incubator 14 can be simultaneously connected, and hence, a well plate holding a virus or a cell can be taken out of the virus storage 13 and the cell culture incubator 14 with sterile conditions kept, and thus, an efficient operation can be performed.

Furthermore, in order to prevent an operation error, the following structure may be employed: With the cell culture incubator 14 used for culturing the T cell, another cell storage including an incubator for holding a CAR-T cell infected with a virus is separately prepared, and this cell storage for the CAR-T cell is connected to the first virus infection isolator 7 in a position different from that of the cell culture incubator 14.

Although the cell storage is configured as an incubator in the above-described example, the cell storage may be a simple storage vessel with the culture station S1 configured as an incubator.

In this case, the culture station S1 and the cell storage are configured to be connectable/disconnectable with connecting means similar to that used for connecting the isolator and the cell storage described above with the sterile conditions kept, and the culture vessel is transferred through the cell storage from each isolator to the culture station S1 for culturing.

Alternatively, the cell storage may be provided with a cleaning filter such as a HEPA filter for attaining ventilation to the outside with the sterile conditions kept inside, and the culture vessel held in the cell storage may be transported to the culture station S1 corresponding to the incubator for culturing the cell.

Similarly, although the nucleic acid storage is configured as a storage in the above-described example, the storage station S2 may be configured as a storage with the nucleic acid storage configured as a simple storage vessel.

In this case, the storage station S2 and the nucleic acid storage are configured to be connectable/disconnectable with connecting means similar to that used for connecting the isolator and the nucleic acid storage described above with the sterile conditions kept, and the culture vessel is transferred through the nucleic acid storage from each isolator to the storage station S2 for storage.

In the description given above, as the procedures for producing the CAR-T cell in the respective isolators, different procedures or operations can be performed as long as the purpose is the production of the CAR-T cell, and accordingly, as each means provided in each isolator, one suitable for the procedures and operations can be used.

### REFERENCE SIGNS LIST

1 genetically modified cell production system
2 cell processing facility
3 material supply isolator
4 cell preparation isolator
5 virus-processing isolator (nucleic acid preparation isolator)
6 cell-processing isolator
7 first virus infection isolator (nucleic acid introduction isolator)
8 second virus infection isolator (nucleic acid introduction isolator)
9 cell collection isolator
11 decontamination device
12 transferring vessel
13 virus storage (nucleic acid storage)
14 cell culture incubator (cell storage)
20 adhesion factor supply device
21 virus supply device
22 activator supply device
23 first seeding device
24 second seeding device
25 third seeding device
26 cell collecting device

1. A genetically modified cell production system for producing a genetically modified cell having a gene modified by introducing a nucleic acid into a cell, comprising:
a cell-processing isolator for seeding a cell in a culture vessel with sterile conditions kept inside, and a cell storage, configured to be connectable/disconnectable to/from the cell-processing isolator with the sterile conditions kept, for holding the culture vessel in which the cell has been seeded to transfer the cell;
a nucleic acid preparation isolator for preparing a nucleic acid to be introduced into the cell with sterile conditions kept inside, and a nucleic acid storage, configured to be connectable/disconnectable to/from the nucleic acid preparation isolator with the sterile conditions kept, for holding the prepared nucleic acid for transfer; and
a nucleic acid introduction isolator, configured to have sterile conditions kept inside, and be connectable/disconnectable simultaneously to/from the cell storage and the nucleic acid storage with the sterile conditions kept, for introducing the nucleic acid having been transported from the nucleic acid storage into the cell having been transported from the cell storage.

2. The genetically modified cell production system according to claim 1, comprising, as the nucleic acid introduction isolator:
a first nucleic acid introduction isolator for first introducing the nucleic acid into the cell having been cultured in the cell storage; and
a second nucleic acid introduction isolator for introducing the nucleic acid again into the cell having been processed in the first nucleic acid introduction isolator.

3. The genetically modified cell production system according to claim 1, comprising a decontamination device for decontaminating an inside of each of the isolators.

4. The genetically modified cell production system according to claim 1, comprising a decontamination station, to which the cell storage in plural number is connectable, for decontaminating an inside of each cell storage connected.

5. The genetically modified cell production system according to claim 1, wherein:
the cell is a T cell;
a virus having a nucleic acid encoding a chimeric antigen receptor (CAR) is prepared in the nucleic acid preparation isolator; and
the nucleic acid is introduced into the T cell in the nucleic acid introduction isolator by infecting the T cell with the virus for producing a CAR-T cell as the genetically modified cell.
